(19) United States
(12) Patent Application Publication
(10) Pub. No.: US 2025/0195411 A1
(43) Pub. Date: Jun. 19, 2025

(54) EYELID DYSFUNCTION TREATMENT SERUM

(71) Applicant: TearRestore, Inc., New Brighton, MN (US)

(72) Inventors: Ken DauSchmidt, Hugo, MN (US); Joseph J. Hendrickson, New Brighton, MN (US); Omar E. Awad, Shoreview, MN (US); Scott G. Hauswirth, Cape Coral, FL (US); Joseph J. Allen, Richmond, VA (US)

(21) Appl. No.: 18/987,326

(22) Filed: Dec. 19, 2024

Related U.S. Application Data
(60) Provisional application No. 63/611,789, filed on Dec. 19, 2023.

(51) Int. Cl. A61K 9/00; A61K 9/107; A61K 31/05; A61K 31/125; A61K 36/53; A61K 36/61; A61K 47/14; A61K 47/32; A61K 47/44; A61P 25/02; A61P 27/02

(52) U.S. Cl. CPC A61K 9/0014; A61K 9/107; A61K 31/05; A61K 31/125; A61K 36/53; A61K 36/61; A61K 47/14; A61K 47/32; A61K 47/44; A61P 25/02; A61P 27/02

(57) ABSTRACT

Various embodiments disclosed relate to a serum for treating eyelid dysfunction. A serum may include a transient receptor potential melastatin 8 (TRPM 8) activator in an amount of 0.10 wt. % to 0.75 wt. % of the serum. A serum may include a carrier oil with dermal permeability in an amount of 0.25 wt. % to 0.50 wt. % of the serum. A serum may include a thickener. A serum may include an emollient. A serum for treating eyelid dysfunction may induce an increase in blink rate of at least 2 blinks per minute and produce an ocular soothing effect.

### PRIORITY

This application claims the benefit of priority to U.S. Provisional Patent Application Ser. No. 63/611,789, filed Dec. 19, 2023, which is incorporated by reference in its entirety.

### BACKGROUND

Meibomian Gland Disease (MGD) is the leading cause of dry eye. MGD occurs when glands in the eyelid, the Meibomian glands, do not release sufficient amounts of the oils needed for a healthy tear film, leading to premature evaporation of the watery layer of the tear film. Patients with MGD can suffer from symptoms such as redness, tearing, burning or gritty sensations, blurry vision, and the like. One of the primary risk factors for developing MGD is a reduced rate of blinking, often associated with extended screen use. It has been well documented that the rate of blinking is reduced significantly when staring at a screen. This reduced blink rate prevents proper distribution of tears across the ocular surface and compromises the natural moisturizing mechanism of the eye. The combination of a reduced blink rate and a non-stabilized tear film can lead to rapid drying to the ocular surface, resulting in discomfort brought about by this rapid drying.

Solutions for managing dry eye symptoms primarily rely on behavioral modifications to increase blink frequency. These approaches typically involve software applications or digital reminders that prompt users to blink at regular intervals through visual or auditory cues. The behavioral methods require constant active user response and do not address the underlying physiological mechanisms of MGD.

### SUMMARY OF THE DISCLOSURE

The present inventors have recognized, among other things, that a problem to be solved can include increasing the blink rate. The present subject matter can help provide a solution to this problem, such as stimulating a neural response through the use of a chemically active serum. The serum can incorporate neuron stimulating compounds that target multiple pathways including activating a transient receptor potential melastatin 8 (TRPM8) cold receptor, stimulating Meibum production from a Meibomian gland to stabilize a tear film stability, or activating a trigeminal nerve to naturally increase blink rates. The serum can include an active ingredient like peppermint oil or menthol, and delivered by a dermal carrier system that enhances absorption into the eyelid tissue. The serum can be applied on or in close proximity to the eyelids. The serum can be composed of a variety of compounds that elicit a blinking response. The eye area can comprise both the upper eyelid and the lower eyelid of the eye. The serum can be combined with a variety of other treatments including thermal energy. The serum can be delivered to the eyelid in a variety of different methods, such as, through an applicator, as an oil, as a paste, in mist form, or through an adhesive patch.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate, by way of example, but not by way of limitation, various examples discussed in the present document.

FIG. 1 depicts an applicator for a serum for treating eyelid dysfunction in an example.

FIG. 2A depicts comparative Ocular Surface Disease Index (OSDI) results of patients using a serum for treating eyelid dysfunction in an example.

FIG. 2B depicts comparative blinks per minute results of patients using a serum for treating eyelid dysfunction in an example.

FIG. 2C depicts comparative partial blinks per minute results of patients using a serum for treating eyelid dysfunction in an example.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Discussed herein are an eyelid treatment serum and associated methods. The serum can include one or more blink stimulating compounds to address eyelid irritation and fatigue caused by Meibomian gland dysfunction (MGD) and prolonged screen use. Dysfunction of the Meibomian gland impairs the production of the protective lipid layer of tears, while extended screen time reduces natural blink rates leading to tear film evaporation, both leading to dry eye symptoms and patient discomfort.

According to various aspects of the present disclosure, a serum for treatment of eyelid dysfunction can deliver active ingredients to the periocular region to increase blink rate in a patient. This increased blink rate can provide a soothing and cooling effect to the patient to mitigate the effects of dry eye from MGD and extended screen use. In some examples, the treatment serum can also be used to treat conditions, such as, dry eye, blepharitis, hordeolum, rosacea, chalazion, Meibomian gland dysfunction, allergies, conjunctivitis, eye fatigue, inflammation of an eyelid, eye trauma, or a combination thereof.

Dry eye syndrome is a condition that occurs when the tear film covering the cornea experiences excessive evaporation, leading to corneal exposure to air. The corneal exposure can result in various uncomfortable symptoms, such as, eye irritation, redness, burning a gritty sensation, blurred vision, or a combination thereof. Dry eye syndrome condition can develop from a combination of contributing factors that affect tear film stability and periocular surface health.

One mechanism that can lead to dry eye involves decreased tear production. The reduction in tear output can be attributed to several underlying causes, such as, an age-related change in lacrimal gland function, a medical condition affecting tear production, a medicinal side effect, a hormonal fluctuation, a previous laser eye surgery procedure, or direct damage to a tear-producing gland.

Increased tear evaporation can represent another pathway in the development of dry eye syndrome. Environmental conditions can play a role in this process, with factors such as wind, low humidity, smoke, or dust exposure contributing to an accelerated tear film evaporation. A particular contributor to increased tear film evaporation is MGD, a chronic condition characterized by a blockage, or an abnormal secretion pattern of a Meibomian gland located in an eyelid. A Meibomian gland normally produce Meibum, an oily substance that forms the protective layer of the tear film. When a gland becomes clogged with a thick secretion or fail to produce adequate amounts of oil, the tear film can become unstable and evaporate more rapidly from an ocular surface.

Other lifestyle factors can impact tear film stability. For example, extended use of a digital screen can lead to a reduced blink rate, which can prevent a proper tear film refreshment and distribution across a corneal surface. The decreased blink rate during screen use can result in prolonged exposure of the tear film to air, accelerating evaporation. Additional factors that can contribute to increased tear evaporation can include contact lens wear, which can interfere with normal tear film dynamics, and Vitamin A deficiency, which can affect the health and function of an ocular surface epithelium.

Unlike conventional behavioral modifications and reminders to increase blinking, the serum formulation takes a physiological approach by incorporating neuron-stimulating compounds that target multiple pathways including TRPM8 cold receptors, Meibomian gland stimulation, and trigeminal nerve activation to naturally increase blink rates and tear film stability. The serum can be capable of exciting receptors in mucous membranes in or near the eyelid. The serum can be capable of stimulating the trigeminal nerve. The serum can stimulate free nerve endings. The serum can cool an eyelid. The serum can activate the TRPM8 cold sensory receptors in the cornea. In some cases, the serum can include an irritant configured to trigger one or more autonomic responses. In some cases, the serum can include one or more compounds configured to elicit muscle contraction or a common chemical sense.

The serum can include a neuron stimulating compound such as a TRPM8 activator. The TRPM8 pathway functions as a specialized sensory system that responds to both cold temperatures and chemical agonists, triggering non-selective cation channels that create a cooling sensation. The incorporation of a TRPM8 activator in the serum can provide direct symptomatic relief through activation of a cold receptor in the periocular region. The activation of the cold receptor can generate a cooling and soothing sensation that helps counteract the irritation and discomfort associated with a dry eye condition. The cooling and soothing sensation can help provide relief to a patient while other therapeutic mechanisms take effect. The cooling sensation can provide comfort and help reduce the inflammatory response often present in the dry eye condition.

The TRPM8 activator can be configured to stimulate receptors in a Meibomian gland to enhance tear film stability through production of Meibum. When activated, the TRPM8 receptor can enhance a production and secretion of Meibum, the oily substance that forms a protective outer layer of a tear film. The increased Meibum production can in turn aid in maintaining the tear film's stability and reduce excessive evaporation of the tear film.

The TRPM8 activator can be configured to stimulate a trigeminal nerve to elicit muscle contraction to increase blink rate. By increasing the blink rate, the serum can help distribute tears more effectively across the ocular surface, stimulate additional tear production, and maintain better tear film stability.

The neuron stimulating compound can include, for example, menthol, eucalyptus, or camphor, among others. The serum can include a carrier oil, such as jojoba or coconut oil. The carrier oil can have dermal permeability when applied to an eyelid. The serum can additionally include one or more soothing or cooling agents, such as an agent or compound that reduces sensory discomfort when applied to an eyelid. In some cases, the serum can include a pharmaceutically active agent, an afferent neural stimulating compound, or other compounds or agents. The serum can be in a form of a solid, a paste, an ointment, a gel, a liquid, an aerosol, a mist, a polymer, a film, an emulsion, or a suspension.

The serum can be applied to an eyelid through an applicator, such as, an applicator sized and shaped for efficient application to a Meibomian gland. The applicator can be made of a pliable material. The method of treatment can include applying the serum to the eyelid on an exterior surface. The serum can be applied with an adhesive or an eyelid wipe. The serum can be applied along with a thermal treatment. In some cases, the thermal treatment can be timed. The serum can be used for treating dry eye, blepharitis, hordeolum, rosacea, chalazion, Meibomian gland dysfunction, allergies, conjunctivitis, trauma, or combinations thereof.

### Definitions

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In the event of inconsistent usages between this document and any documents so incorporated by reference, the usage in this document controls.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substantially free of” as used herein can mean having none or having a trivial amount of, such that the amount of material present does not affect the material properties of the composition including the material, such that about 0 wt. % to about 5 wt. % of the composition is the material, or about 0 wt. % to about 1 wt. %, or about 5 wt. % or less, or less than or equal to about 4.5 wt. %, 4, 3.5, 3, 2.5, 2, 1.5, 1, 0.9, 0.8, 0.7, 0.6, 0.5, 0.4, 0.3, 0.2, 0.1, 0.01, or about 0.001 wt. % or less, or about 0 wt. %.

The term “antimicrobial agent” as used herein refers to a substance that can prevent or inhibit the growth of microorganisms to protect the shelf-life of a product. An antimicrobial agent can be used to prevent or inhibit the growth of bacteria, fungi, or other pathogens. Non-limiting examples of an antimicrobial agent can include an organic acid and their salts, a paraben, an alcohol, a phenol, a natural antimicrobial, a quaternary ammonium compound, or other synthetic preservatives.

The term “antioxidant” as used herein refers to a compound that can prevent oxidative damage by neutralizing free radicals and reactive oxygen species that can cause cellular damage. Non-limiting examples of an antioxidant can include a vitamin-based antioxidant, a polyphenol, an enzymatic antioxidant, a botanical antioxidant, a synthetic oxidant, or a mineral-based antioxidant.

The term “chelating agent” as used herein refers to a compound that can bind to and sequester metal ions to prevent the metal ions from interfering with the stability, efficacy, and safety of a product. A chelating agent can be used to sequester metal ions such as calcium, magnesium, iron, copper, or the like. Non-limiting examples of a chelating agent can include an aminocarboxylic acid, a phosphate, a phosphonate, an organic acid, a natural chelator, a synthetic polymeric chelator, or a hydroxy acid.

The term “emollient” as used herein refers to a substance that can soften and smooth the skin by forming a protective barrier layer on the skin surface. An emollient can have occlusive or humectant properties when applied topically. Non-limiting examples of an emollient can include a natural oil, or a synthetic oil, and can be oil-based or water-based.

The term “emulsifier” used herein refers to a compound that can reduce surface tension between two phases and can act as an emulsifying, dispersing, or wetting agent in a topical formulation. An emulsifier can also be known as a surfactant. An emulsifier contains both a hydrophilic and hydrophobic region to facilitate mixing of water-based and oil-based components. Non-limiting examples of an emulsifier can include an anionic surfactant, a cationic surfactant, a non-ionic surfactant, or an amphoteric surfactant. An emulsifier can be a synthetic or a naturally derived substance.

The term “serum” as used herein refers to a topical formulation that can contain an active ingredient suspended or dissolved in a vehicle designed for penetration and delivery to a tissue layer. The serum can comprise a combination of active ingredients, solvents, and other additives to achieve a therapeutic effect. A serum can include a water-based formulation, an oil-based formulation, or a combination of both. The term “serum,” “treatment serum,” “serum formulation”, as used herein can be used interchangeably and synonymously.

The term “solvent” as used herein refers to a liquid that can dissolve a solid, a liquid, or a gas. Non-limiting examples of a solvent can include a silicone, an organic compound, water, an alcohol, an ionic liquid, or a supercritical fluid.

The term “thickener” as used herein refers to a substance that can increase the viscosity of a formulation. Non-limiting examples of a thickener can include a natural gum, a polysaccharide, a cellulose derivative, a synthetic polymer, clay, minerals, a protein-based thickener, or a modified starch.

### Serum Formulation

An example formulation for a serum is summarized below in Table 1.

TABLE 1
Example serum formulation

| Components | Examples |
|---|---|
| TRPM8 activator | Peppermint |
| | (Mentha arvensis) leaf oil |
| Carrier oil | Jojoba oil |
| Thickener | Polyacrylate crosspolymer-6 |
| Emollient | Coco-caprylate/caprate, squalene |
| Solvent | Water |
| Antioxidant | Green tea |
| | (Camellia sinensis) leaf extract |
| Vitamin | Niacinamide, tocopheryl acetate |
| Antimicrobial agent | Gluconolactone, sodium benzoate, |
| | potassium sorbate, green ta |
| | (Camellia sinensis) leaf extract |
| Chelating agent | Sodium phytate |
| Hydrating agent | Sodium hyaluronate |
| Others - stimulant | Caffeine, coffee (Coffea arabica) seed extract |
| Others - fatty acid | Sacha Inchi (Plukenetia volubilis) seed oil |

The formulation in Table 1 is an example formulation, not an exhaustive list. Alternative or additional components can be included in the formulation. Components in the serum can perform multiple roles and are not limited to one specific function. For example, green tea (Camellia sinensis) leaf extract can be both an antioxidant and an antimicrobial agent.

Transient Receptor Potential Melastatin 8 (TRPM8) Activator. The serum can include one or more TRPM8 activators. A TRPM8 activator can include, but is not limited to, peppermint (Mentha arvensis) oil, menthol, eucalyptus, camphor, icilin, their derivatives, or a combination thereof. In an example, the serum can include a TRPM8 activator from about 0.10 wt. % to about 0.75 wt. % of the formulation, or about 0.10, 0.15, 0.20, 0.25, 0.30, 0.35, 0.40, 0.45, 0.50, 0.55, 0.60, 0.65, 0.70, or about 0.75 wt. % of the formulation.

Carrier Oil. The serum can include one or more carrier oils with dermal permeability to enable delivery of a TRPM8 activator through a skin barrier by facilitating absorption and penetration of the TRPM8 activator into deeper layers of tissue. The carrier oil can improve the overall bioavailability of the formulation, enabling greater access of the components of the serum to their intended target sites. A carrier oil can include, but is not limited to, jojoba (Simmondsia chinensis) oil, coconut oil, argan oil, rosehip oil, grapeseed oil, a caprylic/capric triglyceride, palm kernel oil, evening primrose oil, hemp seed oil, chia seed oil, flaxseed oil, squalene, camellia oil, hazelnut oil, macadamia oil, their derivatives, or a combination thereof. In an example, the serum can include a carrier oil from about 0.25 wt. % to about 2.00 wt. % of the formulation, or about 0.25, 0.50, 0.75, 1.00, 1.25, 1.50, 1.75, or about 2.00 wt. % of the formulation.

Thickener. The serum can include one or more thickeners or gelling agent. A thickener or gelling agent can aid in the viscosity of the serum, tack of the serum, or coverage of the serum upon application. For example, a thickener or gelling agent in the formulation can provide consistency to the serum and improve a flow characteristic of the serum to enable smooth spreading of the serum on the skin. This increased viscosity can improve formulation stability of the serum, improve adherence of the serum to the skin, and retard evaporation of the serum after application. A thickener or gelling agent can include, but are not limited to, polyacrylate crosspolymer-6, a xanthan gum, a cellulose derivative, a carrageenan, an alginate, a gum, a polyacrylamide, a hydroxyethylcellulose, a polyethylene glycol (PEG) derivative, an acrylate copolymer, hydroxypropyl methylcellulose, sodium carboxymethylcellulose, a modified starch, a crosspolymer dimethicone, dimethicone, a dimethiconal mixture, or a combination thereof. In some examples, the thickener can be a polymeric thickener. The treatment serum can include a thickener from about 0.10 wt. % to about 1.00 wt. % of the formulation, or about 0.10, 0.12, 0.14, 0.16, 0.18, 0.2, 0.22, 0.24, 0.25, 0.26, 0.28, 0.30, 0.32, 0.34, 0.36, 0.38, 0.40, 0.42, 0.44, 0.46, 0.48, 0.50, 0.75 or about 1.00 wt. % of the formulation.

In some examples, the serum can be formulated to have a viscosity of 15 mPa·s to 30 mPa·s through the addition of a thickener. This viscosity can be measured using a Brookfield DV-II+ Pro Digital Viscometer of its equivalent. The measurement can be conducted at 25° C. at a relative humidity of 40-60%, using a LV spindle. The viscometer can be operated at a rotational speed of 60 rpm. The viscosity can be measured 30 seconds after turning on the viscometer. Three consecutive measurements can be taken at 1-minute intervals, and the average of the measurements can be reported as the final viscosity value in cp or mPa·s.

Emollient. The serum can include an emollient to form a skin barrier layer to prevent moisture loss and hydrate the skin. An emollient can be used to fill in the spaces between skin cells with lipids, increasing hydration, and reducing water loss through evaporation from the skin. An emollient can improve skin texture and smoothness through this moisturization. An Emollient can include, but is not limited to, jojoba oil, argan oil, squalene, coco-caprylate/caprate, rosehip oil, evening primrose oil, dimethicone, cyclomethicone, isopropyl myristate, caprylic triglyceride, cetyl alcohol, shea butter, cocoa butter, or a combination thereof. In an example, the serum can include an emollient from about 0.01 wt. % to about 5.00 wt. % of the formulation, or about 0.01, 0.25, 0.50, 0.75, 1.0, 1.25, 1.5, 1.75, 2.0, 2.25, 2.50, 2.75, 3.00, 3.25, 3.50, 3.75, 4.00, 4.25, 4.50, 4.75, or about 5.00 wt. % of the formulation.

Solvent. The serum can include a solvent to serve as a carrier and a delivery vehicle for an active ingredient. The solvent can ensure proper dispersion, dissolution, consistency, and absorption of components of the serum. A solvent can include, but is not limited to, water, ethanol, propylene glycol, butylene glycol, glycerin, pentylene glycol, hexylene glycol, polyethylene glycol, propanediol, ethoxydiglycol, or the like. The serum can include a combination of solvents. In an example, the solvent can be about 20 wt. % to about 99 wt. % of the formulation, or about 20, 25, 30, 35, 40, 45, 50, 55, 60, 65, 70, 75, 80, 85, 90, 95, or about 99 wt. % of the formulation.

Antioxidant. The serum can include one or more antioxidants. Antioxidants can prevent degradation of the serum through neutralization of free radicals and reactive oxygen species. The presence of an antioxidant in the serum can extend the shelf life of the serum. An antioxidant can be included in the serum to impart an antioxidant property to the patient. An antioxidant can include, but is not limited to, L-ascorbic acid (vitamin C), tocopherol (vitamin E), retinol (vitamin A), niacinamide (vitamin B3), panthenol (vitamin B5), green tea (Camellia sinensis) extract, resveratrol, ferulic acid, grape seed extract, butylated hydroxytoluene, butylated hydroxyanisole, propyl gallate, or the like. The serum can include an antioxidant from about 0.01 wt. % to about 2.00 wt. % of the formulation, or about 0.01, 0.10, 0.20, 0.30, 0.40, 0.50, 0.60, 0.70, 0.80, 0.90, 1.00, 1.10, 1.20, 1.30, 1.40, 1.50, 1.60, 1.70, 1.80, 1.90, or about 2.00 wt. % of the formulation.

Vitamin. The serum can include one or more vitamins. A vitamin can promote skin health, provide essential nutrients for skin cells, support collagen production, enhance the skin barrier, or a combination thereof. A vitamin can include, but is not limited to, L-ascorbic acid (vitamin C), niacinamide (vitamin B3), panthenol (vitamin B5), cyanocobalamin (vitamin B12), retinol (Vitamin A), tocopherol or tocopheryl acetate (vitamin E), cholecalciferol (vitamin D), phytonadione (vitamin K), or their derivatives. The serum can include a vitamin from about 0.01 wt. % to about 5.00 wt. % of the formulation, or about 0.01 0.20, 0.40, 0.60, 0.80, 1.00, 1.20, 1.40, 1.60, 1.80, 2.00, 2.20, 2.40, 2.60, 2.80, 3.00, 3.20, 3.40, 3.60, 3.80, 4.00, 4.20, 4.40, 4.60, 4.80, or about 5.00 wt. % of the formulation.

Antimicrobial agent. The serum can include one or more antimicrobial agents. A microbial agent can prevent microbial growth, maintain product safety, extend the shelf life of the serum, or a combination thereof. An antimicrobial agent can also be called a preservative. An antimicrobial agent can be delivered to the skin of the patient to control acne-causing bacteria or balance the skin microbiome balance. An antimicrobial agent can include, but is not limited to, triclosan, chlorhexidine, benzalkonium chloride, imidazolidinyl urea, tea tree oil, green tea (Cameillia sinensis) leaf extract, thyme oil, oregano extract, neem extract, caprylyl glycol, ethylhexylglycerin, phenoxyethanol, sodium benzoate, gluconolactone, potassium sorbate, or the like. The treatment serum can include an antimicrobial agent from about 0.01 wt. % to about 5.00 wt. % of the formulation, or about 0.01, 0.50, 1.00, 1.50, 2.00, 2.50, 3.00, 3.50, 4.00, 4.50, or about 5.00 wt. % of the formulation.

Chelating agent. The serum can include one or more chelating agents. A chelating agent can bind metal ions to prevent formulation destabilization, prevent oxidative reactions, extend the shelf life, or a combination thereof. A chelating agent can include, but is not limited to, sodium phytate, ethylenediaminetetraacetic acid (EDTA), tetrasodium EDTA, disodium EDTA, sodium gluconate, trisodium ethylenediamine disuccinate, citric acid, gluconic acid, sodium phytate, tartaric acid, sodium hexametaphosphate, pentasodium pentetate, trisodium nitrilotriacetate (NTA), or the like. The serum can include a chelating agent from about 0.01 wt. % to about 0.50 wt. % of the formulation, or about 0.01, 0.02, 0.04, 0.06, 0.08, 0.10, 0.12, 0.14, 0.16, 0.18, 0.20, 0.22, 0.24, 0.26, 0.28, 0.30, 0.32, 0.34, 0.36, 0.38, 0.40, 0.42, 0.44, 0.46, 0.48, or about 0.50 wt. % of the formulation.

Hydrating agent. The serum can include one or more hydrating agents. A hydrating agent can attract and bind water to the skin, maintain skin hydration levels, improve skin elasticity, or a combination thereof. A hydrating agent can include, but is not limited to, hyaluronic acid, glycerin, sodium pyrrolidone carboxylic acid (PCA), urea, propylene glycol, lactic acid, sodium lactate, aloe vera, sodium hyaluronate, hydrolyzed collagen, urea, or the like. The serum can include a hydrating agent from about 0.01 wt. % to about 2.00 wt. % of the formulation, or about 0.01, 0.20, 0.40, 0.60, 0.80, 1.00, 1.20, 1.40, 1.60, 1.80, or 2.00% of the formulation.

Emulsifier. The serum can include an emulsifier to aid in mixing of a water phase and an oil phase. An emulsifier is particularly suitable when the serum is in the form of an emulsion or if immiscible materials are being combined. An emulsifier can also include surfactants. The emulsifier can have micellar properties, lamellar properties, or both. An emulsifier can be nonionic, anionic, zwitterionic, or cationic. An emulsifier can include, but is not limited to, a polysorbate, a PEG derivative, a ceteareth compound, decyl glucoside, a laureth compound, cocamidopropyle betaine, sodium cocoamphoacetate, disodium cocoamphodiacetate, lauryl betaine, lecithin, a saponin, cetyl PEG/PPG-10/1 dimethicone, lauryl polyglyceryl-3 polydimethylsiloxyethyl dimethicone, a soy-based surfactant, a sugar-based surfactant, or the like. In an example, the emulsifier can be about 3.0 wt. % to about 8.0 wt. % of the formulation, or about 3.0, 3.5, 4.0, 4.5, 5.0, 5.5, 6.0, 6.5, 7.5, or about 8.0 wt. % of the formulation.

Other additives can also be included in the serum. For example, a filler can be used to bulk up the formulation, a pigment or a dye can be added to modify color, a fragrance compound or an essential oil can be added to impart a scent, or a combination thereof. A filler can include, but is not limited to, silica, kaolin clay, talc, mica, titanium dioxide, microcrystalline cellulose, corn starch, rice powder, tapioca starch, bamboo powder, polymethyl methacrylate, polyethylene, silicone powder, or the like. A pigment or dye can include, but are not limited to, iron oxides, titanium dioxide, zinc oxide, chromium oxide, ultramarine, beta carotene, carmine, turmeric, or the like. A fragrance compound or essential oil can include, but are not limited to, an aldehyde, an ester, an ionone, a synthetic floral, lavender oil, rose oil, citrus oils, jasmine oil, sandalwood oil, or the like. Other additives can include caffeine, coffee (Coffea arabica) seed abstract, ginger (Zingiber officinalis) root extract, or fatty acids like Sacha Inchi (Plukenetia volubilis) seed oil. In some examples, the serum can include a wax, a powder, an electrolyte component, an ultraviolet (UV) protector, a fatty alcohol, a skin conditioning agent, or the like.

In some examples, the serum can be pH controlled to improve product stability, improve skin compatibility, or a combination thereof. The pH control can be achieved through the addition of an acid or a base. An acid can include, but is not limited to, citric acid, lactic acid, gluconic acid, or the like. A base can include, but is not limited to sodium hydroxide, triethanolamine, aminomethyl propanol, or the like. In some examples, a buffer system can be included in the serum to maintain pH stability during the serum's shelf life to prevent a pH drift that can compromise the formulation's stability or effectiveness. A buffer solution can include, but is not limited to, a citric acid and sodium citrate system, a lactic acid and sodium lactate system, or the like.

### Method of Making Serum Formulation

The instantly described serum can be made according to a variety of suitable methods. For example, solvent can be added to a blending vessel. A thickener can be added to the solvent in the blending vessel and mixed until the solution or suspension is free from large solids. Next, a hydrating agent can be added to the solution or suspension and mixed until the hydrating agent is dissolved. The other ingredients can subsequently be added, including, a chelating agent, a vitamin, a preservative, an antioxidant, an emollient, a fatty acid, or an additive. In some examples, the described formulations can be made in a batch manufacturing process, a semi-batch manufacturing process, or a continuous manufacturing process.

### Method of Using

The serum can be contained in an applicator assembly. A variety of conventional applicators are known in the art (e.g., sponges, brushes, wipes, blenders, or other applicators). In some examples, the serum can be applied in a single layer, or in multiple layers for a desired therapeutic effect. In some examples, the serum can be applied using an applicator, an adhesive patch, an eye mask, an eyelid wipe, a cloth, a spray, a mist, a hand, a brush, or a combination thereof.

In some examples, the applicator can be sized and shaped to target a Meibomian gland. The applicator can be constructed from a pliable material to conform to an eyelid's natural contours to enable thorough and gentle coverage of a treatment area. In some examples, the applicator can have a metal tip, a plastic tip, or an elastomeric tip. In some examples, the applicator can have a rolling ball tip.

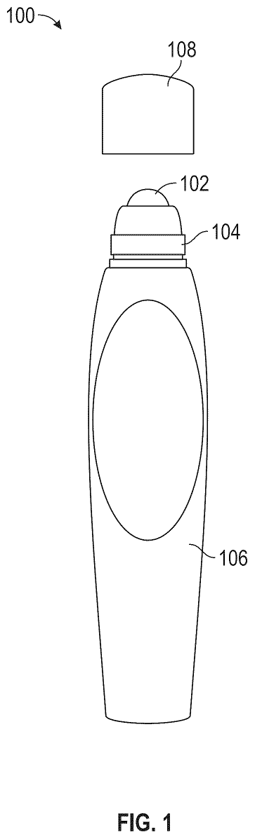

FIG. 1 depicts an applicator for a serum for treating eyelid dysfunction in an example. The applicator 100 can include a rolling ball 102 that aids in applying the serum onto the patient. The rolling ball 102 can be in fluid contact with a reservoir 106 which houses the serum. When the rolling ball 102 is applied to the patient's eyelid, the serum can be transferred from the reservoir 106 to an area of application on the patient's eyelid due to the rolling motion of the rolling ball 102. The applicator 100 can include a locking collar 104 to allow a cap 108 to be secured to a body or reservoir 106 of the applicator. The locking collar can include a threaded connection system, a snap fit joint, a bayonet mounting system, a press-fit mechanism, a friction-fit mechanism, ribbed surfaces, elastomeric liners, a push-and-turn design, a squeeze-and-turn design, or a combination thereof. In some examples, the rolling ball 102 can be made of metal or plastic. In some examples, the reservoir 106 can be made of metal or plastic.

In some examples, the serum can be applied using an adhesive-based delivery system that allows the serum to remain in contact with a treatment area for an extended period. The adhesive-based delivery system can be in the form of a patch that can conform to the shape of an eyelid and can be applied to a lower eyelid or an upper eyelid.

In some examples, the serum can be administered using an eye mask delivery system that provides controlled release of the serum. The eye mask delivery system can combine the delivery of the serum with a heat treatment therapy. The combination with heat therapy can help improve the penetration of the serum during application.

In some examples, the serum can be delivered via a fine mist or spray applicator that provides a uniform coverage across an eyelid surface. The fine mist or spray applicator can have a directional or an adjustable nozzle to direct and vary the coverage of the serum during application. The fine mist or spray applicator can allow a patient to use a non-contact delivery system to deliver the serum to a sensitive area.

In some examples, the serum can be applied directly to the eyelid through a manual application method, such as, by hand, an eyelid wipe, a brush, or a combination thereof. Manual application of the serum can be combined with cleaning of the eyelid surface and massage to the eyelid.

In an example, a method of treating eyelid dysfunction can include cleansing a periocular area of the patient prior to applying the serum, applying a serum to an upper eyelid of the user, inducing an increase in a blink rate of at least 2 blinks per minute and producing an ocular soothing effect, and allowing the serum to dry on the upper eyelid for at least 30 seconds. In some examples, the method of treating eyelid dysfunction can include applying the serum to a lower eyelid of the user and allowing the serum to dry on the lower eyelid for at least 30 seconds.

### EXAMPLES

Various examples of the present disclosure can be better understood by reference to the following Examples, which are offered by way of illustration. The present disclosure is not limited to the Examples give herein.

### Example 1: Ocular Surface Disease Index (OSDI)

A Control sample and Sample A was tested on 30 participants to determine the effectiveness of the serum for treating eyelid dysfunction based on an OSDI test. The OSDI test is a questionnaire used to assess symptoms of ocular disease and their impact on vision-related functioning. The administering of the OSDI test was to determine if the application of the serum improved ocular disease symptoms in participants when compared to the control sample.

A Control was prepared by adding 97.2 g of water to a blending vessel and heated to 25° C. Next, 0.4 g of Sepimax Zen (a polyacrylate crosspolymer) was slowly added into the blending vessel and mixed until all clumps were dispersed. Next, 2.1 g of Geoguard Ultra (a gluconolactone and sodium benzoate mixture) was added into the mixture and stirred until dissolved. Lastly, 0.2 g of potassium sorbate was added into the mixture and stirred until dissolved. Table 2 shows the types and amounts of components of the control formulation.

TABLE 2
Control formulation.

| Components | Examples | Source | Amount (wt. %) |
|---|---|---|---|
| Solvent | Water | On-site | 20.00-99.00 |
| Thickener | Sepimax Zen | Lotion crafter | 0.10-0.50 |
| Antimicrobial | Geoguard | Formulator | 0.01-5.00 |
| agent | ultra | Sample | |
| | | Shop | |
| Antimicrobial | Potassium | Formulator | 0.01-5.00 |
| agent | sorbate | Sample | |
| | | Shop | |

Sample A was prepared by adding 90.95 g of water to a blending vessel and heated to 25° C. Next, 0.4 g of Sepimax Zen (a polyacrylate crosspolymer) was slowly added into the blending vessel and mixed until all clumps were dispersed. Next, 0.1 g of sodium hyaluronate was slowly added into the blending vessel and mixed until all clumps were dispersed. Next, 0.1 g of sodium phytate, 1 g of niacinamide, 2 g of Geoguard Ultra (a gluconolactone and sodium benzoate mixture), 0.2 g of potassium sorbate, 0.1 g of green tea (Camillia sinensis) leaf extract, 2 g of coco-caprylate/caprate, 0.25 g of caffeine, were added to the blending vessel individually and sequentially, and mixed until all solids were dissolved. 1 g of jojoba oil, 1 g of squalene, 0.5 g of Sacha Inchi (Plukenetia volubilis) seed oil, 0.05 g of Vitamin E acetate, 0.05 g of coffee (Coffea arabica) seed extract, 0.05 g of ginger (Zingiber officinalis) root extract, and 0.25 g of peppermint (Mentha arvensis) leaf oil were added to the blending vessel individually and sequentially, and mixed until all solids were dissolved. Sample A contains the TRPM8 active ingredient in the form of the peppermint (Mentha arevenis) leaf oil, which is absent in the Control. Table 3 shows the types and amounts of the components of Sample A.

TABLE 3
Sample A formulation.

| Components | Examples | Source | Amount (wt. %) |
|---|---|---|---|
| Solvent | Water | On-site | 20.00-99.00 |
| Thickener | Sepimax Zen | Lotion crafter | 0.10-0.50 |
| Hydrating agent | Hyaluronic Acid | Making Cosmetics | 0.01-2.00 |
| | EcoCert | | |
| Chelating agent | Sodium phytate | Formulator Sample | 0.01-0.50 |
| | | Shop | |
| Vitamin | Niacinamide | Formulator Sample | 0.01-5.00 |
| | | Shop | |
| Vitamin | Vitamin E Acetate | Jedwards | 0.01-5.00 |
| Antimicrobial agent | Geoguard ultra | Formulator Sample | 0.01-5.00 |
| | | Shop | |
| Antimicrobial agent | Potassium sorbate | Formulator Sample | 0.01-5.00 |
| | | Shop | |
| Antioxidant | Green tea extract FSS | Formulator Sample | 0.01-2.00 |
| | PF | Shop | |
| Emollient | Coco- | Formulator Sample | 0.01-5.00 |
| | Caprylate/Caprate | Shop | |
| Emollient | Squalene | Jedwards | 0.01-5.00 |
| Caffeine | Caffeine USP | Ingredients to Die | 0.01-0.50 |
| | anhydrous | For | |
| Carrier oil | Jojoba oil | Jedwards | 0.25-2.00 |
| Fatty acid | Sacha Inchi oil | Jedwards | 0.01-2.00 |
| Additive | Coffee bean CO2 | Eden Botanicals | 0.01-2.00 |
| | Extract | | |
| Additive | Ginger CO2 Extract | Eden Botanicals | 0.01-2.00 |
| TRPM8 Activator | Peppermint Japanese | Essential Natural Oil | 0.10-0.75 |
| | EO Organic | | |

The OSDI test was measured for 30 participants at baseline (before serum application) and 15 minutes after application of the serum. The study design included a group that was administered the Control, and a group that was administered Sample A, with participants randomly assigned to each group. The OSDI questionnaire was administered by a clinical researcher by asking each participant 12 questions about their eye symptoms at the baseline and after 15 minutes of the eyelid serum treatment. The OSDI questionnaire assesses dry eye symptoms and the effects of dry eye symptoms have on vision-related function in the past week of the participant's life. Questions in the OSDI questionnaire were divided into 3 main categories of ocular symptoms (e.g., light sensitivity, eye pain, irritation in the eyes, etc.), vision-related functionality (e.g., reading etc.), and environmental triggers (e.g., exposure to dry environments or air conditioned areas). Participants rated their responses on a 0 to 4 scale with 0 corresponding to “none of the time” and 4 corresponding to “all of the time” A final score was calculated which ranges from 0 to 100, with scores of 0 to 12 representing normal eye conditions, 13 to 22 representing mild dry eye disease, 23 to 32 representing moderate dry eye disease, and greater than 33 representing severe dry eye disease.

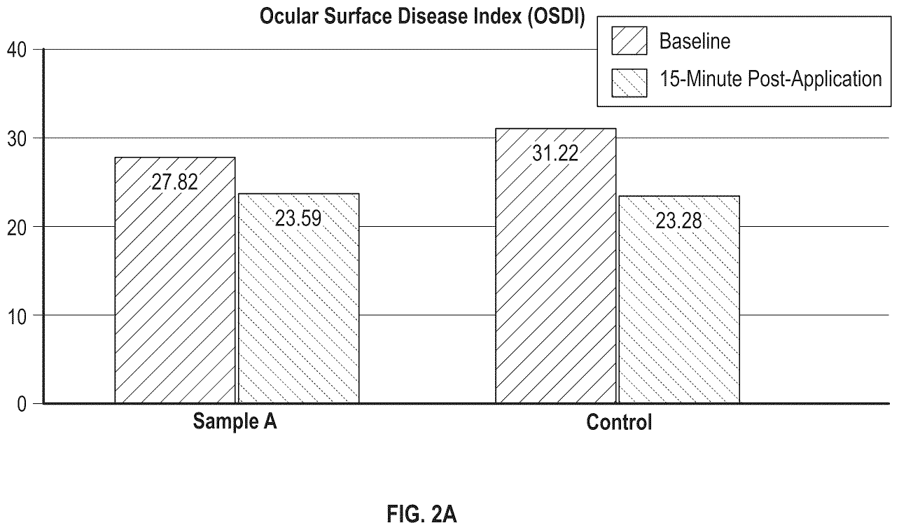

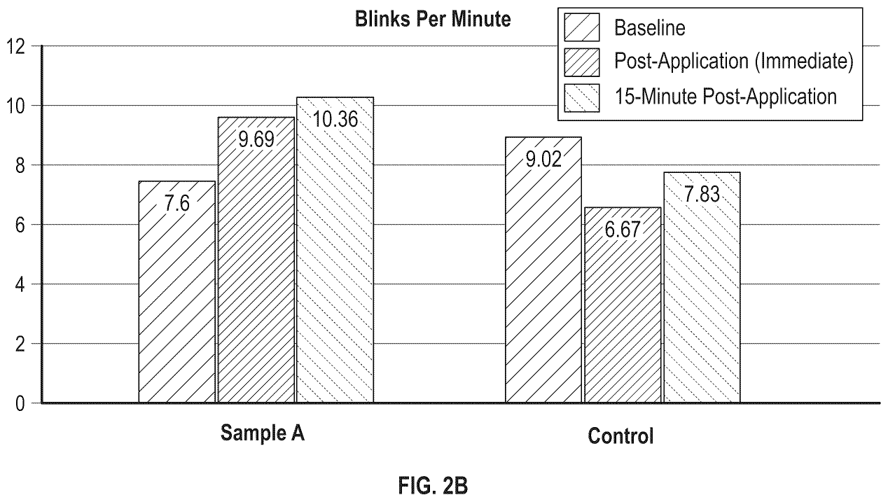

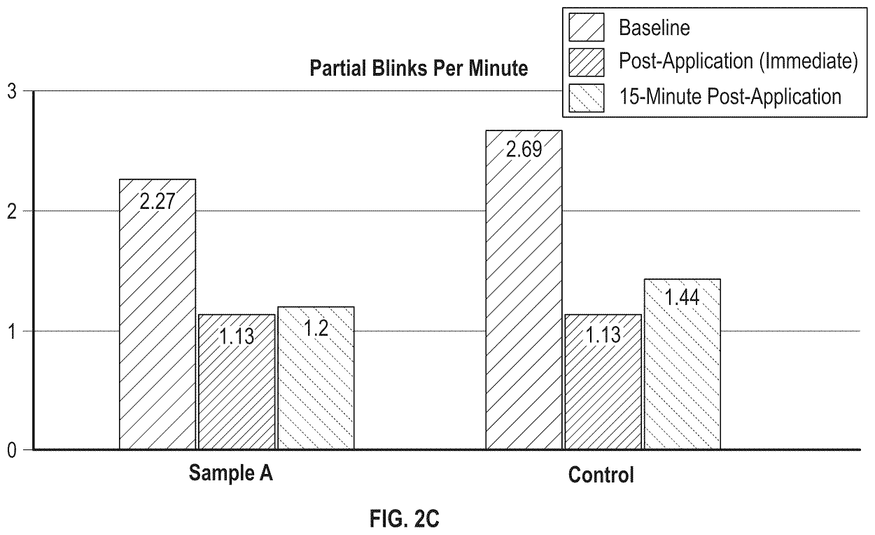

FIG. 2A depicts comparative Ocular Surface Disease Index (OSDI) results of patients using a serum for treating eyelid dysfunction in an example. The results of the OSDI tests are summarized below in Table 4:

TABLE 4
OSDI results.

| Test type | Time of measurement | Control | Sample A |
|---|---|---|---|
| OSDI | Baseline | 31.22 | 27.82 |
| | 15 mins after application | 23.28 | 23.59 |

FIG. 2A and table 4 show that the participants' OSDI was reduced from an average of 27.82 to 23.59 in Sample A, representing a 15% decrease after the use of the serum. This shows that the serum can be used to reduce dry eye symptoms by at least 15%. The results indicate that after application of the serum, participants' experience reduced symptoms of ocular surface disease within a brief time of 15 mins. The reduced symptoms can be translated to lowered irritability and increased comfort of the participant.

### Example 2: Blink Rate

Control and Sample A were tested on 30 participants to determine the effectiveness of the serum formulation based on blink rate, with the blink rate being indicative of the comfort of a patient. A higher blink rate can correspond to better lubrication of the eyes due to increased distribution of tears across the ocular surface. An increased blinking action can also help regenerate and spread the tear film more frequently, which can improve corneal wetting and maintain better ocular surface health. An improved corneal wetting and increased blink rate can lead to improved comfort for the patient.

Blink rate per minute was measured for 30 participants at baseline (before serum application), immediately after application of the serum, and 15 mins after application of the serum. The study design included a group that was administered the Control, and a group that was administered Sample A, with participants randomly assigned to each group. The measurement was collected using manual observation over a 5 minute interval while the participant was reading a text. The total number of blinks was recorded on video and counted. The total number of blinks and partial blinks were divided by 5 to obtain the blink rate per minute.

FIGS. 2B and 2C depict comparative blinks per minute and partial blinks per minute results of patients using a serum for treating eyelid dysfunction in an example. The results of the blink test and the partial blink test are summarized below in Table 5:

TABLE 5
Blink test and partial blink test results

| Test type | Time of measurement | Control | Sample A |
|---|---|---|---|
| Blink Rate | Baseline | 9.02 | 7.6 |
| (min⁻¹) | Immediately after application | 6.67 | 9.69 |
| | 15 mins after application | 7.83 | 10.36 |
| Half Blink Rate | Baseline | 2.69 | 2.27 |
| (min⁻¹) | Immediately after application | 1.13 | 1.13 |
| | 15 mins after application | 1.44 | 1.2 |

Table 5 shows the use of the serum in Sample A demonstrated an increase in blink rate from 7.6 blinks per minute to 9.69 blinks per minute immediately after application of the treatment serum. The increase translates to a 27% increase in blink rate immediately after application of the serum when compared to the baseline. Table 5 also shows that the blink rate increases from 7.6 blinks per minute to 10.36 blinks per minute 15 minutes after application of the serum. The increase translates to a 36% increase in blink rate 15 minutes after application of the treatment serum when compared to the baseline. This shows that the serum can be used to increase the blink rate by at least 30%. This increase shows that the serum induces an increase in blink rate of at least 2 blinks per minute and produces an ocular soothing effect. In contrast, the control shows a decrease in blink rate immediately and 15 minutes after the application. For example, the Control shows that the blink rate decreased from 9.02 blinks per minute, to 6.67 blinks per minute immediately after application of Control. The blink rate continued to decrease 15 minutes after the application of Control. Therefore, the results in Table 5 and FIG. 2B-2C show that the serum in Sample A is more effective at addressing eyelid irritation, increasing eye comfort, stimulating the natural blink reflex, and aiding in sustained ocular comfort. Sample A's ability to maintain elevated blink rates over time, when compared to the Control, validates the serum's therapeutic efficacy in promoting optimal ocular surface health and sustained comfort to the patient.

### VARIOUS NOTES & EXAMPLES

In some aspects, the techniques described herein relate to a serum for treating eyelid dysfunction including: a transient receptor potential melastatin 8 (TRPM8) activator in an amount of 0.10 wt. % to 0.75 wt. % of the serum; a carrier oil with dermal permeability in an amount of 0.25 wt. % to 0.50 wt. % of the serum; a thickener; and an emollient; wherein the serum induces an increase in blink rate of at least 2 blinks per minute and produces an ocular soothing effect.

In some aspects, the techniques described herein relate to a serum, wherein the transient receptor potential melastatin 8 (TRPM8) activator comprises peppermint oil, menthol, eucalyptus, camphor, or combinations thereof.

In some aspects, the techniques described herein relate to a serum, wherein the carrier oil comprises jojoba oil, coconut oil, or combinations thereof.

In some aspects, the techniques described herein relate to a serum, wherein the thickener comprises a polymeric thickener.

In some aspects, the techniques described herein relate to a serum, wherein the polymeric thickener comprises polyacrylate crosspolymer-6.

In some aspects, the techniques described herein relate to a serum, wherein the polyacrylate crosspolymer-6 comprises 0.25 wt. % to 1.00 wt. % of the serum.

In some aspects, the techniques described herein relate to a serum, wherein the emollient comprises squalene, coco-caprylate/caprate or combinations thereof.

In some aspects, the techniques described herein relate to a serum, wherein the emollient comprises 0.50 wt. % to 3.00 wt. % of the serum.

In some aspects, the techniques described herein relate to a serum, wherein the transient receptor potential melastatin 8 (TRPM8) activator is configured to stimulate receptors in a Meibomian gland to enhance tear film stability through production of Meibum.

In some aspects, the techniques described herein relate to a serum, wherein the transient receptor potential melastatin 8 (TRPM8) activator is configured to stimulate a trigeminal nerve to elicit muscle contraction to increase blink rate.

In some aspects, the techniques described herein relate to a serum, wherein the serum is in a form of a solid, a paste, a gel, a liquid, an aerosol, a mist, a film, an emulsion, or a suspension.

In some aspects, the techniques described herein relate to a serum, wherein the serum comprises an antioxidant, a vitamin, an antimicrobial agent, a chelating agent, a hydrating agent, or combinations thereof.

In some aspects, the techniques described herein relate to a method of treating eyelid dysfunction including: applying a serum to an upper eyelid of a user, the serum comprising a transient receptor potential melastatin 8 (TRPM8) activator in an amount of 0.10 wt. % to 0.75 wt. % of the serum and a carrier oil with dermal permeability in an amount of 0.25 wt. % to 0.50 wt. % of the serum; and inducing an increase in a blink rate of at least 2 blinks per minute and producing an ocular soothing effect.

In some aspects, the techniques described herein relate to a method, further including cleansing a periocular area of the user prior to applying the serum; and allowing the serum to dry on the upper eyelid for at least 30 seconds.

In some aspects, the techniques described herein relate to a method, further including applying the serum to a lower eyelid of the user, and allowing the serum to dry on the lower eyelid for at least 30 seconds.

In some aspects, the techniques described herein relate to a method, wherein applying the serum includes using an applicator, an adhesive patch, an eye mask, an eyelid wipe, a cloth, a spray, a mist, a hand, a brush, or a combination thereof.

In some aspects, the techniques described herein relate to a method wherein the applicator includes a metal tip, a plastic tip, or a rolling ball.

In some aspects, the techniques described herein relate to a method, wherein the serum is used for treating dry eye, blepharitis, hordeolum, rosacea, chalazion, Meibomian gland dysfunction, allergies, conjunctivitis, eye fatigue, inflammation of an eyelid, eye trauma, or combinations thereof.

In some aspects, the techniques described herein relate to a method, wherein the serum is used to reduce dry eye symptoms by at least 15%.

In some aspects, the techniques described herein relate to a method, wherein the serum is used to increase the blink rate by at least 30%.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A serum for treating eyelid dysfunction, the serum comprising:
a transient receptor potential melastatin 8 (“TRPM8”) activator in an amount of 0.10 wt. % to 0.75 wt. % of the serum;
a carrier oil with dermal permeability in an amount of 0.25 wt. % to 0.50 wt. % of the serum;
a thickener; and
an emollient;
wherein the serum induces an increase in blink rate of at least 2 blinks per minute and produces an ocular soothing effect.

2. The serum of claim 1, wherein the TRPM8 activator comprises peppermint oil, menthol, eucalyptus, camphor, or combinations thereof.

3. The serum of claim 1, wherein the carrier oil comprises jojoba oil, coconut oil, or combinations thereof.

4. The serum of claim 1, wherein the thickener comprises a polymeric thickener.

5. The serum of claim 4, wherein the polymeric thickener comprises polyacrylate crosspolymer-6.

6. The serum of claim 5, wherein the polyacrylate crosspolymer-6 comprises 0.25 wt. % to 1.00 wt. % of the serum.

7. The serum of claim 1, wherein the emollient comprises squalene, coco-caprylate/caprate or combinations thereof.

8. The serum of claim 1, wherein the emollient comprises 0.50 wt. % to 3.00 wt. % of the serum.

9. The serum of claim 1, wherein the TRPM8 activator is configured to stimulate receptors in a Meibomian gland to enhance tear film stability through production of Meibum.

10. The serum of claim 1, wherein the TRPM8 activator is configured to stimulate a trigeminal nerve to elicit muscle contraction to increase blink rate.

11. The serum of claim 1, wherein the serum is in a form of a solid, a paste, a gel, a liquid, an aerosol, a mist, a film, an emulsion, or a suspension.

12. The serum of claim 1, wherein the serum further comprises an antioxidant, a vitamin, an antimicrobial agent, a chelating agent, a hydrating agent, or combinations thereof.

13. A method of treating eyelid dysfunction comprising:
applying a serum to an upper eyelid of a user, the serum comprising a transient receptor potential melastatin 8 (“TRPM8”) activator in an amount of 0.10 wt. % to 0.75 wt. % of the serum and a carrier oil with dermal permeability in an amount of 0.25 wt. % to 0.50 wt. % of the serum; and
inducing an increase in a blink rate of at least 2 blinks per minute and producing an ocular soothing effect.

14. The method of claim 13, further comprising:
cleansing a periocular area of the user prior to applying the serum; and
allowing the serum to dry on the upper eyelid for at least 30 seconds.

15. The method of claim 14, further comprising:
applying the serum to a lower eyelid of the user, and
allowing the serum to dry on the lower eyelid for at least 30 seconds.

16. The method of claim 14, wherein applying the serum includes using an applicator, an adhesive patch, an eye mask, an eyelid wipe, a cloth, a spray, a mist, a hand, a brush, or a combination thereof.

17. The method of claim 16, wherein the applicator includes a metal tip, a plastic tip, or a rolling ball.

18. The method of claim 13, wherein the serum is used for treating dry eye, blepharitis, hordeolum, rosacea, chalazion, Meibomian gland dysfunction, allergies, conjunctivitis, eye fatigue, inflammation of an eyelid, eye trauma, or combinations thereof.

19. The method of claim 13, wherein the serum is used to reduce dry eye symptoms by at least 15%.

20. The method of claim 13, wherein the serum is used to increase the blink rate by at least 30%.
